(19) United States
(12) Patent Application Publication
(10) Pub. No.: US 2019/0060108 A1
(43) Pub. Date: Feb. 28, 2019

(54) COOLING WRISTBAND

(71) Applicant: Maggie Ferrari, Carmichael, CA (US)

(72) Inventor: Maggie Ferrari, Carmichael, CA (US)

(21) Appl. No.: 15/690,429

(22) Filed: Aug. 30, 2017

(51) Int. Cl. A61F 7/02; A61F 7/10

(52) U.S. Cl. CPC A61F 7/02; A61F 2007/0035; A61F 2007/023; A61F 7/10

(57) ABSTRACT

A cooling wrist band that includes an outer surface; an inner surface, where the inner surface includes inner pockets; and plurality of cold compress packets within the inner pockets. The plurality of cold compress packets may include individual packets place into individual inner pockets. In one embodiment, the plurality of cold compress packets include individual packets.

### BACKGROUND OF THE INVENTION

### Field of Invention

The present invention relates to a wristband, which provides cooling sensations.

### Description of Related Art

Hot flashes are a common condition that affect both men and women but is especially common in middle-aged women as they experience menopause. A hot flash is an intense feeling of heat and sweating with a rapid heartbeat that lasts for a short period of time. The hot flash is principally due to hormonal changes in women as they experience menopause. The intense heat with sweating and rapid heartbeat is uncomfortable and usually causes problems for many women. Some women have more intense hot flashes than others and some may suffer multiple hot flashes during the day. The hot flashes can affect the individual's sleep, mood, concentration and cause other physical problems. Any type of relief is welcome for many sufferers of hot flashes.

As a result, it would be advantageous to have a solution in the form of application of a cold sensation, which could help alleviate some of the affects of hot flashes. Application of a cold pack such as a cold pack over the skin could be helpful in addressing to reduce the effects of a hot flash.

### SUMMARY OF THE INVENTION

The present invention relates to a cooling wrist band that includes an outer surface; an inner surface, where the inner surface includes inner pockets; and plurality of cold compress packets within the inner pockets. The plurality of cold compress packets may include individual packets place into individual inner pockets. In one embodiment, the plurality of cold compress packets include individual packets place into a single inner pocket and the plurality of cold compress packets include up to six individual packets.

### BRIEF DESCRIPTION OF DRAWINGS

FIG. 1 depicts a wristband and cold compress packets used in conjunction with the wrist band in accordance with the present invention.

FIG. 2 depicts implementation of the cool packs placed within a wristband.

### DETAILED DESCRIPTION

The present invention relates to a cooling wristband that is applied over the wrist area of a user. The wristband includes an inner surface with inner pockets that receive cold compress packets that may be activated as needed by the user. Preferably, a plurality of cooling packets is placed within the inner surface pockets of the wristband. These cooling packets may be depressed to activate the cooling sensation. So, a user may depress a single packet or multiple packets as needed to alleviate hot flash symptoms.

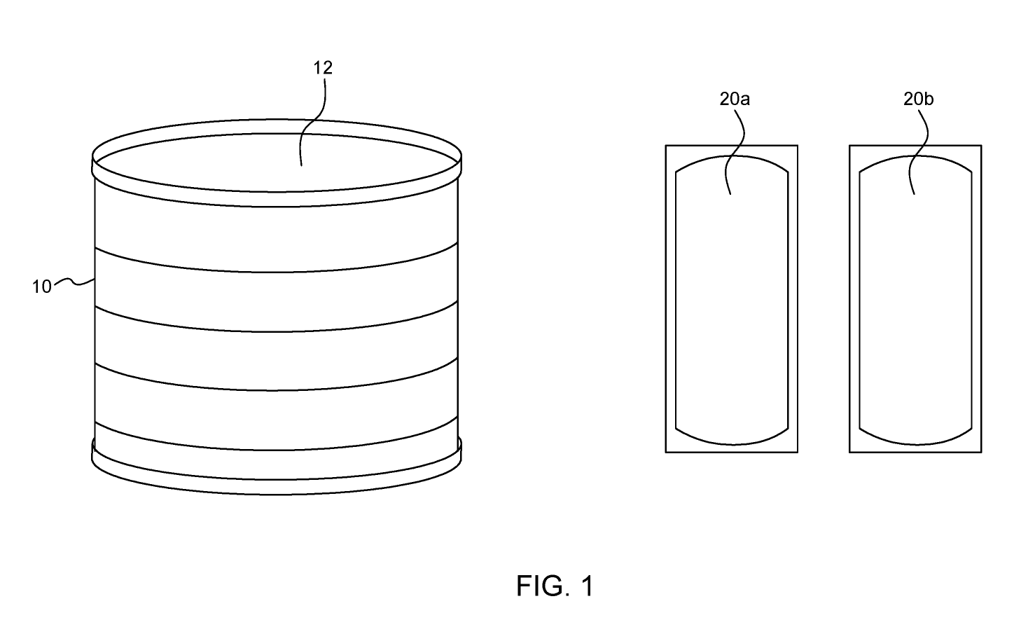

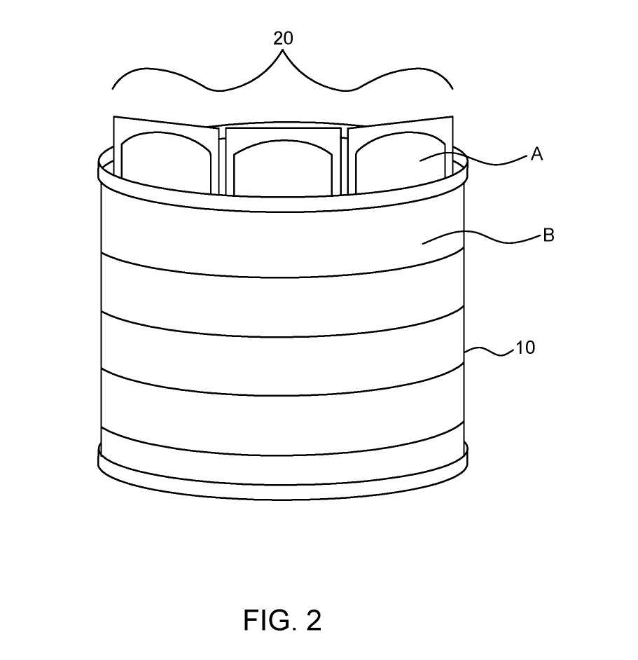

In reference to FIG. 1, a wristband 10 is shown that in accordance with the present invention includes inner pockets 12. The inner pockets 12 receive cold compress packets 20a, 20b as shown in FIG. 1. These compress packets 20a, 20b are depressed in order to activate a cooling sensation as needed. In reference to FIG. 2, a series of cooling packets 20 are inserted into the wristband 10. Therefore, a user has the cooling packets available on the surface of the skin during use. The present invention as a result provides a simple but yet effective manner to addressing relief for hot flashes. It's easily worn by a user and easily activated by depression of the cold packets within the wristband.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The exemplary embodiment was chosen and described in order to best explain the principles of the invention and its practical application, to thereby enable others skilled in the art to best utilize the invention and various embodiments with various modifications as are suited to the particular use contemplated.

What is claimed is:

1. A cooling wrist band comprising;
a outer surface;
an inner surface, where the inner surface includes inner pockets; and
plurality of cold compress packets within the inner pockets.

2. The cooling wrist band according to claim 1, where the plurality of cold compress packets include individual packets place into individual inner pockets.

3. The cooling wrist band according to claim 1, where the plurality of cold compress packets include individual packets place into a single inner pocket.

4. The cooling wrist band according to claim 1, where the plurality of cold compress packets include up to six individual packets.
